(19) United States
(12) Patent Application Publication
(10) Pub. No.: US 2022/0381775 A1
(43) Pub. Date: Dec. 1, 2022

(54) ANALYTE DETECTION AND QUANTIFICATION BY DISCRETE ENUMERATION OF PARTICLE COMPLEXES

(71) Applicant: Genotix Biotechnologies Inc., Sunnyvale, CA (US)

(72) Inventors: Yong Qin Chen, Coral Gables, FL (US); George C Brittain, Palmetto Bay, FL (US); Yaping Zong, San Jose, CA (US)

(73) Assignee: Genotix Biotechnologies Inc., Sunnyvale, CA (US)

(21) Appl. No.: 17/772,133

(22) PCT Filed: Oct. 28, 2020
(86) PCT No.: PCT/US2020/057794

§ 371 Date: Apr. 27, 2022

Related U.S. Application Data
(60) Provisional application No. 62/926,679, filed on Oct. 28, 2019.

(51) Int. Cl. G01N 33/543; G01N 33/574

(52) U.S. Cl. CPC G01N 33/54313; G01N 33/57434

(57) ABSTRACT

Described herein are systems and methods for the discrete detection and quantification of target analytes in a sample based on their binding by two or more particles to form analyte-linked particle complexes. The analyte-linked particle complexes can be differentiated and enumerated versus unbound singlet particles based on the unique physical characteristics of the particles utilized. In some embodiments of the current invention, this may involve one type of analyte, while in other embodiments it may involve multiple different types of analytes, either individually or in analyte complexes.

### TECHNICAL FIELD

The present disclosure relates generally to the technical field of analyzing and quantifying biological analytes and, more particularly, to systems and methods therefor that provide improved accuracy and sensitivity, greater dynamic range, and simplified work flow.

### BACKGROUND ART

High-sensitivity analytical measurements are fundamental to modern science and medicine. Protein and other small-particle measurements are also broadly essential to biomedical research and medical diagnostics. However, most biomedical assays lack the sensitivity and precision to accurately measure single analytes, particularly within complex sample matrices. Most protein and small-particle assays are formatted around bulk analyses, and often require signal amplification. While bulk analyses can and do provide useful information regarding the system overall, they generally always have fundamental limitations that prevent their ability to identify and quantify multiple characteristics of analytes or analyte subpopulations. This issue can be particularly problematic with complex samples, such as serum, urine or saliva, where the concentrations of target proteins and other analytes are extremely low, there are many non-target analytes present at concentrations that are several orders of magnitude higher than the target analyte, and yet the precision and accuracy of the data or diagnostic readout is essential.

The most commonly used biomedical assays for quantifying specific proteins include enzyme-linked immunosorbent assays (ELISAs), western blotting, and mass spectrometry. ELISAs function by capturing a target analyte with a specific antibody coated to the surface of a plate or a micro bead, and then labeling the analyte with a specific secondary, enzyme-conjugated antibody that allows for subsequent signal amplification—generally pigmentation, luminescence or fluorescence—in order to detect the antibody-analyte sandwich over a range of concentrations. Typically, ELISAs only provide a limited dynamic range, require extensive sample purification and washing, produce analog data that must be compared to calibration curves, and inevitably produce variable results due to even small changes in incubation times, lot-to-lot variation in reagent quality, loss of the analytes or antibodies due to washing and changes in the binding equilibria at different concentrations, slight differences in experimental conditions like temperature or light, the precision of the concentrations of the many substrate and buffer components that are required to produce the signal, and even differences in the stabilities of the various assay components over time in storage. In most cases, ELISAs have a lower limit of detection (LOD) in the pM range, while it is said that most relevant biomedical analytes are present in the serum in the aM to fM range, and they may be orders of magnitude lower in urine and saliva. Increasing the incubation times for the multiple steps to >10 hrs each, dramatically increasing the capture surface area, and/or using fluorogenic or radioisotope-labeled substrates has been shown to have the potential to lower the LOD down to the zeptomolar (zM) range. However, this is not a practical workflow for diagnostic assays, particularly for point-of-care-type environments or any scenario where there are many patient samples and/or time is of the essence.

Methods like western blotting and mass spectrometry are more qualitative, but quantitation can be attempted by correlating the signal intensities to reference standards. This can be sufficient in some circumstances, particularly where more quantitative methods have not been developed, but it is not a precise approach for diagnostic purposes. These methods are actually pivotal to the development and optimization of ELISAs, though ELISAs are fine-tuned for more precise analytical quantification.

A more recent technological development for single-analyte quantification is the digital ELISA. This method utilizes antibody-coated micro beads in a particular ratio sufficient to capture a single target analyte on a subfraction of the beads, and then, similar to a conventional ELISA, labels the target analytes with enzyme-conjugated detection antibodies. The difference from a conventional ELISA, and what makes the assay digital, is that the individual beads, which optimally capture at most 1 protein, are then strewn into separate femtoliter (fL)-sized wells of a 50K- to 200K-well array (called a single-molecule array), and—after exposure to the enzymatic substrate—are discretely counted by both fluorescence and light microscopy. Since the beads are captured within fL-sized wells, the volume surrounding each individual bead is sufficiently small that it enables the enzymatic signal development from even extremely small concentrations of detection enzyme to increase to the point that positive wells can be differentiated from negative wells. Each bead in a fluorescent well is designated as active, and the percentage of active beads is calculated relative to the overall bead count, as determined by light microscopy.

Patent Cooperation Treaty (“PCT”) published patent application WO 2007/098148 A2 entitled “Methods and Arrays for Target Analyte Detection and Determination of Target Analyte Concentration in Solution” discloses both arrays of single molecules and methods of producing an array of single molecules for defined volumes between 10 attoliters and 50 picoliters. The disclosed method enables detecting and quantitating single molecules for biomolecules such as enzymes for discovering function, detecting binding partners or inhibitors, and/or measuring rate constants. The digital counting, with clear differentiation of 0 vs. 1 or more protein, greatly enhances the sensitivity of protein detection by reducing the noise barrier of a conventional ELISA. Unfortunately, the finite number of micro wells in the digital ELISA limits the dynamic range of the assay. In addition, the enzyme concentration in the assay must be carefully balanced in order to minimize background noise and prevent signal saturation.

One major issue for all ELISA-based methods is that the reagents have to be supplied in excess to the target-analyte concentrations in order to produce consistent labeling. For this reason, a wash process must be used after each labeling step to remove the excess, yet ubiquitous, unbound reagents and, thus, minimize the resulting interference to the final measurements. Washing steps not only consume time and resources, but they also change the equilibrium established between bound and free reagents, which could result in bias and the loss of labeled analytes. In addition, enzymatic amplification may result in variable signal outputs for a variety of reasons, including differences in the enzyme kinetics, nonspecific activity, the precise reagent quality or quantity, lot-to-lot variation, and age-related loss of function.

Therefore, there exists a need in the art for improved analyte quantification, with increased accuracy and sensitivity, greater dynamic range, and a simplified workflow.

### DISCLOSURE OF INVENTION

The present disclosure provides systems and methods to improve the analysis and quantification of biological analytes having improved accuracy and sensitivity, greater dynamic range, and a simplified work flow.

In the primary embodiment of this disclosure, a system and a method are proposed for detecting and enumerating one type of target analytes in a sample at the single-analyte level. The system consists of two distinguished groups of particles: capture particles and detection particles. The capture particles are conjugated with an analyte-specific reagent that has a specific affinity to a certain binding site or epitope of the target analyte. The detection particles are conjugated with another analyte-specific reagent that has a specific affinity to a secondary binding site or epitope of the same target analyte. When a sample containing the target analyte is mixed with the capture and detection particles, analyte-linked particle complexes may form. If the concentration of the target analyte in the sample mixture is significantly lower than the concentration of the capture and detection particles, then the complexes will be mostly particle doublets, consisting of a single capture particle linked to a single detection particle through a single target analyte. By analyzing the sample mixture in a manner that enables the discrete detection, differentiation and enumeration of the analyte-linked particle doublets versus the non-analyte-bound, henceforth referred to as unbound, singlet particles, the concentration of the target analyte in the sample may be accurately determined.

At higher target-analyte concentrations, higher-order particle complexes containing multiple analytes may form. By analyzing the sample mixture in a manner that enables the discrete detection, differentiation and enumeration of all analyte-linked particle multiplets versus unbound singlet particles, the concentration of the target analyte in the sample may again be accurately determined.

Note that the analyte specificity of the reagents discussed in this disclosure should not be taken literally. The capture particles in this disclosure may be conjugated to a collection of reagents, C, and the detection particles conjugated to another collection of reagents, D, with a portion of C targeting a group of analytes, A, and a portion of D targeting either A or a subgroup of A. It should also be noted here that C and D may be identical, or partially overlapping, or totally different.

In the preceding embodiment, as well as in subsequent embodiments, of this disclosure, it should be apparent to those of ordinary skill in the art that the particular nomenclature for which a particle is referenced, such as capture or detection particle, is used to simplify this disclosure's explanation. In fact, both types of particles can be inter-changed in both form and identity, and the system can instead be considered to comprise two or more groups of particles (e.g., Group 1, Group 2, etc.) that are capable of forming analyte-linked particle complexes that are discretely distinguishable from unbound singlet particles.

In another embodiment of this disclosure, a system and a method are proposed for multiplexed assays to simultaneously detect and enumerate multiple types of target analytes in a sample at the single-analyte level. The system comprises two distinct groups of particles: capture particles and detection particles, with the capture particles further divided into subgroups, wherein each subgroup of capture particles is uniquely labeled with certain physical characteristics. The capture particles are each conjugated with an analyte-specific reagent that has a specific affinity to a certain binding site or epitope of a particular type of target analyte. The detection particles are each conjugated with one of a different set of analyte-specific reagents that has a specific affinity to a secondary binding site or epitope of their respective type of target analyte. When a sample containing target analytes is mixed with the capture and detection particles, multiple groups of analyte-linked particle complexes may form, each associated with one type of analyte and the corresponding detection particles and subgroup of capture particles. By differentiating and grouping according to the capture-particle labels, multiple types of analytes in a sample can be simultaneously analyzed, with each type of analyte analyzed in the same way as proposed in the primary embodiment of this disclosure.

For those of ordinary skill in the art, it should be apparent that the multiplexed-assay embodiment of this disclosure may also be implemented by differentially labeling the detection particles or both the capture and detection particles.

In a related embodiment of this disclosure, the previously proposed multiplexed-assay embodiment may be combined with one or more conventional analog assays. For example, one subgroup of analyte-specific detection particles—corresponding to one subgroup of labeled capture particles—may be replaced by analyte-specific molecular probes, such as the analyte-specific detection reagents directly conjugated with fluorescent molecules. As a result, the concentration of the corresponding analyte will be extracted from the mean fluorescence intensity of the analyte-linked particle-and-molecular-probe sandwiches, instead of from the enumeration of analyte-linked particle complexes. One possible application of such a combined multiplexed assay is to simultaneously measure, in one sample, low-abundance analytes using analyte-linked particle complexes, and high-abundance analytes using analyte-linked particle-and-molecular-probe sandwiches. It should be apparent to those of ordinary skill in the art that this example can be extended to assays that combine multiple types of particle-conjugated and molecular detection reagents.

In yet another embodiment of this disclosure, a system and a method studies analyte-analyte interactions in a sample at the single-analyte level. The system consists of two distinct groups of particles: capture particles and detection particles. The capture particles are conjugated with an analyte-specific reagent that has a specific affinity to a certain binding site or epitope of a primary target analyte. The detection particles are conjugated with an analyte-specific reagent that has a specific affinity to a certain binding site or epitope of a secondary target analyte. When a sample containing the two analytes is mixed with the capture and detection particles, analyte-linked particle complexes may form due to interaction between the two different target analytes. The analyte complex may, therefore, be analyzed in the same way as in the primary embodiment of this disclosure. In some embodiments of this disclosure, another group of detection particles specific to the primary target analyte may also be introduced to simultaneously measure its concentration, and consequently the fractional occupancy. In yet other embodiments of this disclosure, one or more of the target analytes may be directly or indirectly bound or conjugated to the capture and/or detection particles without intermediating capture and/or detection reagents, or the secondary target analyte may actually be a capture and/or detection reagent. It should be apparent to those of ordinary skill in the art that the analyte-analyte-interaction-assay embodiment of this disclosure may also be multiplexed, as discussed in the previous multiplexed-assay embodiments of this disclosure.

While the proposed embodiments of this disclosure focus on measuring the concentration of analytes or analyte complexes through the discrete detection and enumeration of analyte-linked particle complexes, it should be apparent to those of ordinary skill in the art that they can be easily implemented as kinetics and/or dynamics assays to study analyte-reagent or analyte-analyte interactions, including changes due to the introduction of a modulating agent, such as drugs, pharmaceuticals, proteins, kinases, transcription factors, peptides, sugars, oligosaccharides, polysaccharides, nucleic acids, lipids, detergents, hormones, growth factors, cytokines, chemokines, activators, inhibitors, small-molecule activators, small-molecule inhibitors, other modulators, and/or combinations or complexes thereof. Such assays may be used for optimizing pharmaceutical development, determining drug efficacies and selection, elucidating on-target versus off-target responses, identifying effective concentrations in different experimental and physiological conditions, elucidating pharmacodynamics, mapping signaling and metabolic pathways, optimizing antibody manufacturing, and many other research and/or development applications.

The capture and detection particles, as well as the analyte-linked particle complexes discussed in the various embodiments of this disclosure, may be distinctively characterized using any particle-counting techniques that leverage their distinguishable size, mass, chemical, optical, electrical, magnetic, radioisotopic, and/or biological properties. For example, particles with unique optical properties, such as light scattering, absorption and/or fluorescence, may be distinctively counted using multi-parameter particle counters, such as flow cytometers as described by Howard M. Shapiro in Practical Flow Cytometry, 4^{th} ed. (Wiley, 2003), or imaging or laser-scanning microscopes. Particles with unique sizes may be distinctively counted using impedance-based particle counters. Alternatively, any measurement technique may be utilized that enables the extraction, from a sample, of the distinctive counts of the capture and detection particles, as well as the analyte-linked particle complexes described in the various embodiments of this disclosure.

In this disclosure's various embodiments, analyte refers to any test molecule or particle of interest, including, but not limited to: proteins, kinases, transcription factors, antibodies, receptors, peptides, cytokines, chemokines, sugars, oligosaccharides, polysaccharides, DNA, RNA, oligonucleotides, polynucleotides, nucleotide complexes, single-stranded nucleic acid sequences, double-stranded nucleic acid sequences, natural polymers, synthetic polymers, lipids, detergents, hormones, growth factors, micelles, liposomes, lipoproteins, extracellular vesicles, exosomes, oncosomes, viruses, virus-like particles, cells, cell fragments, natural particles, synthetic particles, synthetic compounds, plant-derived compounds, animal-derived compounds, chemicals, drugs, pharmaceuticals, activators, inhibitors, small-molecule activators, small-molecule inhibitors, modulators, and/or combinations or complexes thereof. In some embodiments of this disclosure, the analytes may be targeted to bind to the capture and/or detection particles by cognate capture and/or detection reagents that are conjugated to the particle surface, while in other embodiments the analytes may be directly or indirectly bound or conjugated to the capture and/or detection particles without using intermediating capture and/or detection reagents, such as by use of covalent bonding or affinity tags. The various embodiments of this disclosure are not limited to any particular analyte.

In this disclosure's embodiments, the capture and detection reagents may be anything that binds to a site or epitope of the target analytes, including, but not limited to: antibodies, binding proteins, peptides, polypeptides, protein complexes, sugars, oligosaccharides, polysaccharides, DNA, RNA, oligonucleotides, polynucleotides, nucleotide complexes, single-stranded nucleic acid sequences, double-stranded nucleic acid sequences, aptamers, natural polymers, synthetic polymers, pharmaceuticals, drugs, lipids, detergents, micelles, liposomes, lipoproteins, extracellular vesicles, exosomes, oncosomes, viruses, virus-like particles, cells, cell fragments, chemicals, etc. In some embodiments of this disclosure, one or more of the capture and/or detection reagents may function as target analytes, particularly in analyte-reagent- and/or analyte-analyte-interaction assays. In some embodiments of this disclosure, the binding may be specific, and in other embodiments the binding may be intentionally nonspecific. In some embodiments of this disclosure, the capture and/or detection reagents may be directly conjugated to the capture and/or detection particles, while in other embodiments the capture and/or detection reagents may be indirectly conjugated to the capture and/or detection particles, such as by use of affinity tags. The various embodiments of this disclosure are not intended to be limited to any particular capture or detection reagent.

In this disclosure's embodiments, affinity tags refer to any molecule or element with affinity to, or that can be identified and more generally targeted by, a second molecule or binding partner, and can be used to bring two components together into a complex when differentially conjugated to the pair of components. Such affinity tags include, but are not limited to: biotin, streptavidin, avidin, neutravidin, hemagglutinin, poly-histidine, maltose-binding protein, myc, glutathione-s-transferase, FLAG, protein A, protein G, protein L, DNA, RNA, oligonucleotides, polynucleotides, single-stranded nucleic acid sequences, double-stranded nucleic acid sequences, etc. In other embodiments of this disclosure, if there is a difference in species origin for a particular pair of capture and/or detection reagents, or some general difference that allows for differentiation between the reagents, for example mouse vs. rabbit antibodies, IgG vs. IgM, IgG1 vs. IgG3, etc., then these differences may also be targeted by species-, class- or isotype-specific targeting reagents, such as an anti-mouse-IgG3-specific antibody, conjugated directly or indirectly to the capture and/or detection particles, functioning similar to affinity tags. In this context, and for the embodiments of this disclosure, species-, class- and isotype-specific targeting reagents and/or antibodies constitute affinity tags and/or reagents. In some embodiments of this disclosure, affinity tags may be used to conjugate the capture and/or detection reagents to the capture particles, the detection particles, both particles, or neither particle. In other embodiments of this disclosure, affinity tags may be used to specifically or non-specifically bind the analyte directly to the capture and/or detection particles, in which scenario a particular capture and/or detection reagent may not be necessary. The various embodiments of this disclosure are not limited to any particular affinity tag or reagent.

Finally, in these embodiments of this disclosure, the capture and detection particles may be composed of any inorganic, organic or biological material, or composite of materials, including, but not limited to: polystyrene, silica, glass, metals, magnets, proteins, peptides, polypeptides, protein complexes, sugars, oligosaccharides, polysaccharides, DNA, RNA, oligonucleotides, polynucleotides, nucleotide complexes, single-stranded nucleic acid sequences, double-stranded nucleic acid sequences, aptamers, natural polymers, synthetic polymers, lipids, detergents, micelles, liposomes, lipoproteins, extracellular vesicles, exosomes, oncosomes, viruses, virus-like particles, cells, cell fragments, etc. The capture and detection particles may also be labeled with certain unique physical, chemical and/or biological characteristics. Furthermore, the capture and detection particles may be of any size, shape, or material uniformity, as long as they can be discretely detected and enumerated. In some embodiments of this disclosure, the particles will be 5 nm to 100 μm in diameter. In other embodiments of this disclosure, the particles will be 2 nm to 10 μm in diameter. In yet other embodiments of this disclosure, the particles will be 5 nm to 2 μm in diameter. In the preferred embodiment of this disclosure, the particles will be 10 nm to 1 μm in diameter.

### BRIEF DESCRIPTION OF DRAWINGS

FIG. 1 is a schematic representation of the primary embodiment of the analyte-linked particle complexes described in this disclosure.

FIG. 2 are schematic representations of various particles and analyte-linked particle complexes that may form in a single-analyte system.

FIG. 3 are schematic representations of analyte-linked particle complexes that may form in a multiplexed assay of this disclosure.

FIG. 4 depicts an analyte complex formed between two moieties, with one bonded to a capture particle and the other to a detection particle.

FIG. 5 depicts possible data that may be extracted from a system in accordance with this disclosure based on the detection of single-particle light scatter and fluorescence.

FIG. 6 depicts possible data that may be extracted from a system in accordance with this disclosure based on the detection of single-particle fluorescence, using fluorescently labeled capture and detection particles.

FIG. 7 depicts empirical 2D-fluorescence-histogram and enumeration results produced by a particular implementation of this disclosure, using fluorescent capture and detection particles at similar concentrations to analyze low, intermediate, and high concentrations of human PSA.

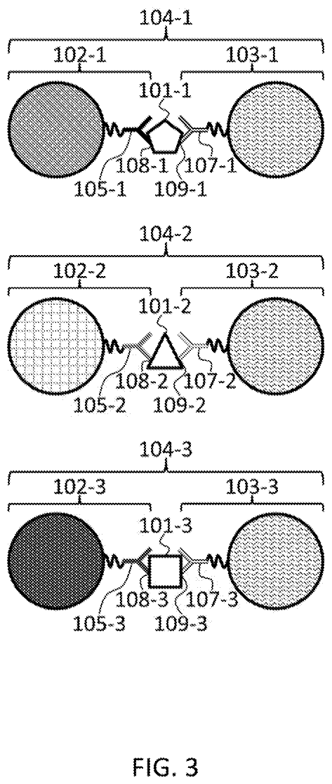

FIG. 3 depicts examples of a gating grid being used to discretely analyze the various analyte-linked particle multiplets formed in 2 fluorescence dimensions, as obtained from a particular implementation of this disclosure, using fluorescent capture and detection particles at similar concentrations to analyze low, intermediate, and high concentrations of human PSA.

FIG. 9 depicts data that may be extracted from a multiplexed assay of this disclosure based on the detection of single-particle light scatter and fluorescence.

FIG. 10 depicts some differences between data that may be extracted using techniques disclosed herein and from some known techniques, comparing the enumeration of particles and analyte-linked particle complexes versus analog signal detection.

FIG. 11 depicts different linear ranges of analyte-linked particle doublets versus higher-order analyte-linked particle complexes, which are shifted to higher analyte concentration ranges.

FIG. 12 illustrates a significant difference between this disclosure's techniques and known techniques, comparing the use of particle-conjugated reagents versus molecular reagents.

### BEST MODE FOR CARRYING OUT THE INVENTION

This disclosure presents systems and methods for the discrete detection and quantification of individual analytes in a sample. More specifically, this disclosure employs binding two particles to target analytes, i.e. a primary analyte-specific capture particle and a secondary analyte-specific detection particle thereby forming analyte-linked particle complexes. In some embodiments of this disclosure, one type of analyte may be targeted by one set of capture and detection particles, while in other embodiments multiple types of analytes may be simultaneously targeted using different subgroups of capture and/or detection particles, each uniquely labeled with certain distinguishable physical characteristics.

In one embodiment of this disclosure, individual target analytes are first bound by a capture particle, and then subsequently bound by a detection particle. In other embodiments of this disclosure the capture and detection particles are added to the sample simultaneously, while in yet other embodiments the sample may be added to a solution containing capture and detection particles.

It will be understood by those skilled in the art that no specific sequence or order in mixing a sample with the particles is critical to this disclosure. Analysis of a sample mixture in a manner that enables the discrete detection, differentiation and enumeration of the analyte-linked particle complexes versus unbound singlet particles, together with differentiating any particle subgroups, can then be used to accurately determine the concentration of one or more types of target analytes in a sample.

In the preferred embodiment of this disclosure, the particles and particle complexes will be analyzed by a multi-parameter particle counter, such as a flow cytometer, or an imaging or laser-scanning microscope. Analyte-linked particle doublets represent the discrete detection of a single analyte, which can be directly counted to give the total number of analytes per volume analyzed. Higher-order particle multiplets discretely shift the fluorescence, light-scatter, and/or other optical signature to greater intensities, representing the discrete inclusion of additional analytes. The total number of analytes counted per volume analyzed can, therefore, be derived from the summation of the number of each type of particle complex multiplied by the corresponding number of analytes contained in each complex. In other words, if Nₐ is the total number of analytes counted per volume analyzed, and Nₐ is the number of counted particle complexes containing m analytes, then

$${N}_{a} = {\sum }_{m} ( m \times {N}_{m} )$$

Furthermore, with sufficient analyte-binding affinity and an excess of capture and detection particles, Nₐ can be directly converted to the analyte molar concentration. At non-equilibrium conditions, or when using capture and/or detection reagents with lower analyte-binding affinities, Nₐ may be compared with a standard curve to determine the analyte molar concentration.

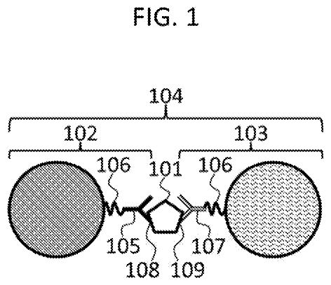

In the primary embodiment of this disclosure, the system and method pertain to the discrete detection and quantification of one type of analyte within a liquid sample matrix. As shown in FIG. 1, this is accomplished by binding the analyte 101 to a primary capture particle 102 and to a secondary detection particle 103, forming an analyte-linked particle doublet 104. The analyte specificity of the capture particle 102 is provided by the specificity of the analyte-specific capture reagent 105, which can be conjugated to the particle by a direct or indirect bond 106, such as by a covalent bond or an affinity tag, including, but not limited to: biotin, streptavidin, hemagglutinin, poly-histidine, glutathione-s-transferase, etc. Similarly, the specificity of the detection particle 103 is provided by the specificity of the analyte-specific detection reagent 107, which can be conjugated to the particle by a direct or indirect bond 106.

A representative capture or detection reagent for protein analytes would be an analyte-specific antibody, which has the characteristic Y-shape depicted in multiple figures of this disclosure. However, the disclosed system is not limited to any particular type of capture or detection reagent. Fundamentally, the capture or detection reagents can be any molecules or entities that bind to the target analytes, specifically or nonspecifically. The capture and detection reagents would preferably bind to the analytes at two different binding sites, shown as 108 and 109 in FIG. 1, although in some cases the experimental objective may be to analyze the competition for two or more binding reagents to a particular binding site. In some embodiments of this disclosure, the capture and/or detection particles may be each targeted to bind to one specific type of analyte, while in other embodiments the capture and/or detection particles may be intended to bind to a mixture of different types of analytes, either specifically or nonspecifically. In the latter case, the specificity of the assay will depend upon the specificity of the detection reagent or reagents.

In this disclosure's figures, the capture 102 and detection particles 103 are each depicted with only one or several capture reagents 105 or detection reagents 107 conjugated to their surface in order to simplify an explanation of the particular embodiment of this disclosure. However, in reality, there could be an unlimited number of capture or detection reagents conjugated to each particle without a particular maximum or minimum number of capture or detection reagents. The precise number of capture or detection reagents will depend on
1. the size of the capture or detection reagents;
2. the size and, thus, surface area of the capture or detection particles;
3. the concentration of the capture or detection reagents used during particle manufacture;
4. the addition of alternative reagents or materials into the manufacturing reaction mixture in order to alter the specific molar ratio of the capture or detection reagents and, thus, compete with them for conjugation to the surface of the capture or detection particles; and
5. other details of specific manufacturing protocols and procedures.
   The preceding constraints would be apparent to those skilled in the art, and it would be clear that the particles depicted in the accompanying drawing FIGs. are simplified for the purpose of explaining this disclosure.

While it may be theoretically possible to conjugate particles with only one copy of a particular reagent, this would actually impose a significant limitation on their functionality in most assays, as it would significantly decrease the probability that an analyte would appropriately contact a capture or detection particle at the location of a capture or detection reagent during any particular collision that occurs between the analytes and the particles in a sample mixture during any interval of time. In contrast, a higher reagent density on the particles means a higher local reagent concentration, which will favor the analyte-reagent bond formation in an equilibrium reaction comparable to a molecular reagent of equivalent concentration that is uniformly distributed in bulk.

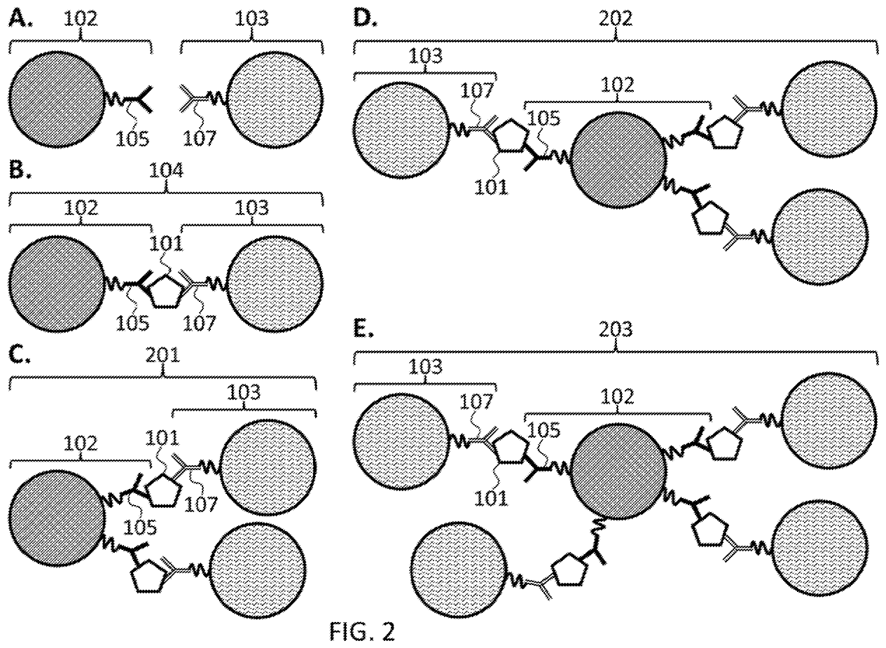

While the primary embodiment of this disclosure describes the formation of analyte-linked particle doublets, each comprising a pair of capture and detection particles bonded together by a single analyte, FIG. 2 illustrates a variety of alternative possibilities that may form or remain in the reaction mixture depending on the concentration ratio of the analytes to the particles. First, as shown in FIG. 2A, if the capture particles 102 and/or detection particles 103 do not bind to an analyte 101, particularly if the concentration of the analyte 101 is lower than the concentrations of the capture and/or detection particles, then there will be residual unbound singlet capture 102 and/or detection particles 103 in the sample mixture. At low analyte concentrations, with sufficient analyte-binding affinity and an excess capture and detection particles, almost all of the target analytes will be present in the form of analyte-linked particle doublets 104, as shown in FIG. 25. If the concentration of the target analytes increases, higher-order analyte-linked particle multiplets will form. FIG. 2C illustrates an analyte-linked particle triplet 201; FIG. 2D an analyte-linked particle quadruplet 202; and, FIG. 2E an analyte-linked particle quintuplet 203. For those skilled in the art, it should be apparent that other types of analyte-linked particle complexes are also possible. Using a multi-parameter particle counter capable of distinguishing these particles and analyte-linked particle complexes from each other, the number of each type of the particles and particle complexes can be easily measured, and the total number of target analytes contained in the sample can be readily extracted from such a measurement, as described previously.

Another embodiment of this disclosure is a multiplexed assay simultaneously targeting multiple different analytes in a complex sample mixture. As depicted in FIG. 3, three different types of analytes 101-1, 101-2, and 101-3 in a sample bond separately to their corresponding capture particles 102-1, 102-2, or 102-3 and detection particles 103-1, 103-2, or 103-3, each one conjugated to a specific capture reagent 105-1, 105-2, or 105-3 or detection reagent 107-1, 107-2, or 107-3 with specificity to a primary binding site 108-1, 108-2, or 108-3 or secondary binding site 109-1, 109-2, or 109-3 on their respective target analyte 101-1, 101-2, or 101-3. In the illustration of FIG. 3, each capture particle 102-1, 102-2, or 102-3, representing a subgroup of capture particles, is labeled with a unique shading, symbolizing the particles possessing some unique physical characteristics and/or labels, such as size and/or optical properties (e.g., intensities and/or wavelengths of absorption, fluorescence and/or light scattering). A multi-parameter particle counter, capable of resolving the particles according to their corresponding labels, can then differentiate and group the particles and analyte-linked particle complexes into subgroups, each corresponding to a specific type of target analyte in the sample mixture. Consequently, each subgroup of capture particles and the corresponding analyte-linked particle complexes can be simultaneously analyzed in the same way as proposed in the primary embodiment of this disclosure, thereby enabling the simultaneous measurement of the concentrations of all three types of target analytes.

It should be apparent to those skilled in the art that FIG. 3 and descriptions thereof in this disclosure are for illustrative purpose only. The multiplexed-assay embodiment of this disclosure is not intended to be limited to any particular format of multiplexed detection, any type of target analyte, or any particular number of different target analytes that are simultaneously analyzed. In some embodiments, this disclosure may be used to analyze 1 to 1000 different target analytes concurrently. In other embodiments, this disclosure may be used to analyze 1 to 100 different target analytes concurrently. In the preferred embodiment, this disclosure is used to analyze 1 to 50 different target analytes concurrently. Increasing the number of multiplexed target analytes may reduce the throughput and dynamic range proportionally, but these can both be counterbalanced and increased by improving the throughput of the instrumentation, or by increasing the acquisition time.

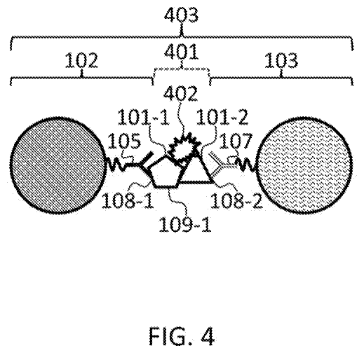

Another embodiment of this disclosure is directed toward studying analyte-analyte interactions. FIG. 4 illustrates one exemplary embodiment of this disclosure wherein the capture particle 102 is targeted to a binding site 108-1 on a primary analyte 101-1, and the detection particle 103 is targeted to a binding site 108-2 on a secondary analyte 101-2, with the primary analyte 101-1 and secondary analyte 101-2 bound together in an analyte complex 401. A modulating agent 402 is also included in FIG. 4 to illustrate actively modulating, inhibiting or enhancing the interaction between the two moieties 101-1 and 101-2 of the analyte complex 401. In the present embodiment of this disclosure, the observation of capture-and-detection-particle complexes 403 represents the binding of the two analytes 101-1 and 101-2 that form the analyte complex 401, and these complexes 401 can be enumerated similar to the enumeration described above for the primary embodiment of this disclosure.

Further, using a variation of the multiplexed assays proposed in the previous embodiments of this disclosure, detection particles labeled with different physical characteristics may be introduced to target the primary analyte 101-1 in order to simultaneously measure its concentration. Such a multiplexed assay would enable the accurate measurement of the fractional occupancy of the secondary analyte 101-2 bound to the primary analyte 101-1 at the single-analyte level. In some cases, the binding of multiple capture and/or detection particles to an analyte complex may be used to identify the presence of multiple copies of the same analyte within the complex.

It should be apparent to those skilled in the art that the proposed embodiment of this disclosure can be used to study, at the single-analyte level, the binding affinities and/or kinetics of two or more analytes that bind together and form analyte complexes, for example, due to the introduction of particular pharmaceuticals, drugs, or other association-modulating molecules or particles of interest. Such assays may be used to optimize pharmaceutical development, determine drug efficacies and selection, elucidate on-target versus off-target responses, identify effective concentrations in different experimental and physiological conditions, elucidate pharmacodynamics, map signaling and metabolic pathways, optimize antibody manufacturing, and many other research and/or development applications. The preceding examples are merely illustrative and not exhaustive, and this disclosure is not limited to any particular format of analyte-complex detection or analysis, any type of target analyte, or any particular number of different target analytes that are concurrently analyzed.

The unbound particles and analyte-linked particle complexes prepared using the systems and methods of this disclosure can be analyzed in any manner that enables the discrete detection, differentiation and enumeration of particle complexes versus singlet particles. In some embodiments of this disclosure, these methods may include, but are not limited to: imaging or laser-scanning microscopy, resistive-pulse sensing, and flow cytometry. In other embodiments of this disclosure, the combination of signals and/or differences in physical properties produced by the proximity of the capture and detection particles in analyte-linked particle complexes can allow for bulk analyses. For example, the size difference between unbound particles and the various analyte-linked particle complexes may be differentiated using centrifuges or size-selective filters, or even dynamic light scattering or nanoparticle tracking analysis. Energy transfer between the capture and detection particles in analyte-linked particle complexes may be interrogated using spectroscopic methods. The preceding list is certainly not exhaustive. Bulk analyses, however, will not provide the precision and accuracy of particle-by-particle counting and analyses. In the preferred embodiment of this disclosure, the particles and analyte-linked particle complexes will be discretely detected, differentiated and enumerated using a multi-parameter particle counter similar to a flow cytometer, or an imaging or laser-scanning microscope.

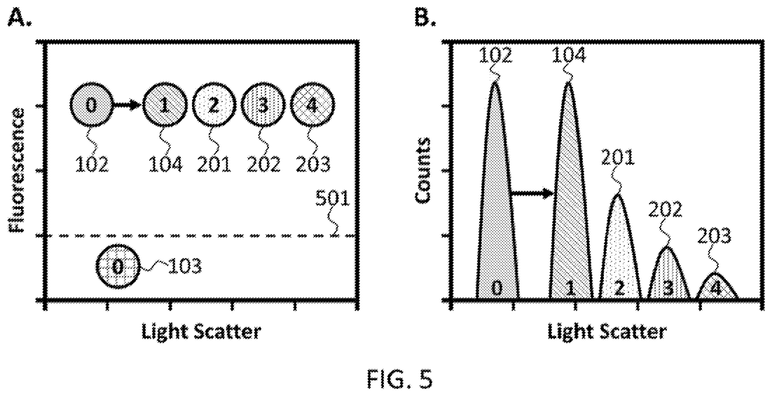

FIG. 5 depicts data that may possibly be extracted from a system in accordance with this disclosure based on the detection of single-particle light scatter and fluorescence, using a multi-parameter particle counter. In this instance, the capture particles are fluorescently labeled, while the detection particles are non-fluorescent. Consequently, in FIG. 5A, the analyte-linked particle complexes 104, 201, 202, and 203 and unbound capture particles 102 are clearly differentiated from unbound detection particles 103 according to their difference in fluorescence intensity. As shown, the fluorescence intensities of the unbound capture particles 102 and all analyte-linked particle complexes 104, 201, 202, and 203 are above the fluorescence threshold 501, while the fluorescence intensity of the unbound detection particles 103 is below the threshold 501.

Furthermore, forming analyte-linked capture-and-detection-particle complexes causes a discrete shift in the light-scatter intensity, dependent on the number of particles bound. FIG. 5B shows a possible histogram plot. After exclusion of the unbound detection particles 103, which are below the fluorescence threshold 501, the number of unbound capture particles 102 and analyte-linked particle complexes 104, 201, 202, and 203, containing correspondingly 0, 1, 2, 3 or 4 detection particles, and thus analytes, can be easily resolved and enumerated.

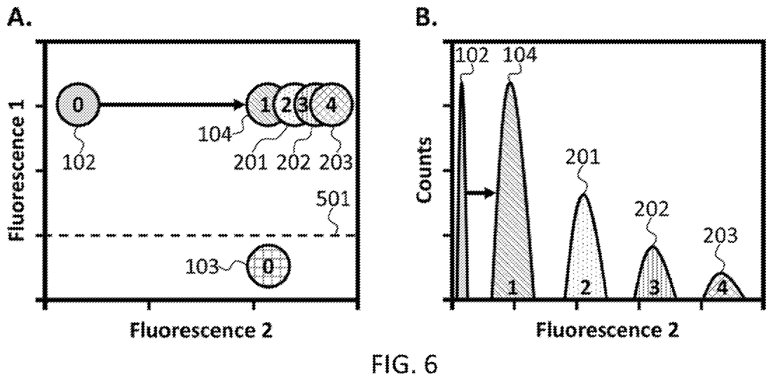

FIG. 6 shows some possible data that may be extracted from a system in accordance with this disclosure based on detecting multi-color single-particle fluorescence, using capture particles that are labeled with a fluorescent color (Fluorescence 1) different from that of detection particles (Fluorescence 2). Consequently, as shown schematically in FIG. 6A, the unbound capture particles 102 and analyte-linked particle complexes 104, 201, 202, and 203 can be clearly differentiated from unbound detection particles 103 according to their difference in the intensity of Fluorescence 1. Meanwhile, forming analyte-linked capture-and-detection-particle complexes causes a discrete shift in the intensity of Fluorescence 2, dependent on the number of analytes bound to capture particles. FIG. 6B shows a possible histogram plot. After exclusion of unbound detection particles 103 with a fluorescence intensity below the threshold 501 in the Fluorescence-1 channel, the number of unbound capture particles 102 and analyte-linked particle complexes 104, 201, 202, or 203 containing 0, 1, 2, 3 or 4 detection particles, and thus analytes, can be easily resolved and enumerated based on their distinct intensities in Fluorescence 2.

Note that the illustrations appearing in FIGS. 5 and 6 are merely illustrative. As stated previously, the types of analyte-linked particle complexes observed in an actual embodiment of this disclosure would depend on the relative concentrations of the analytes to the particles.

### INDUSTRIAL APPLICABILITY

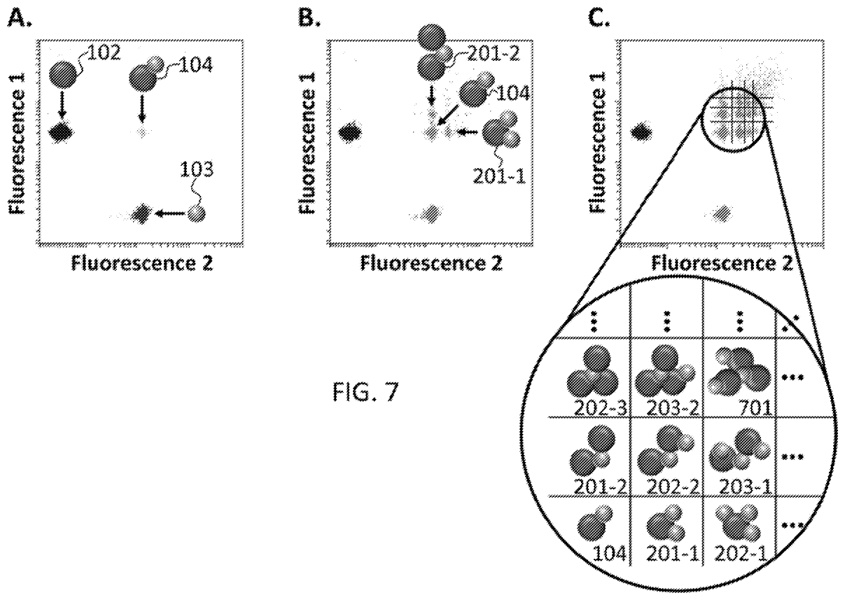

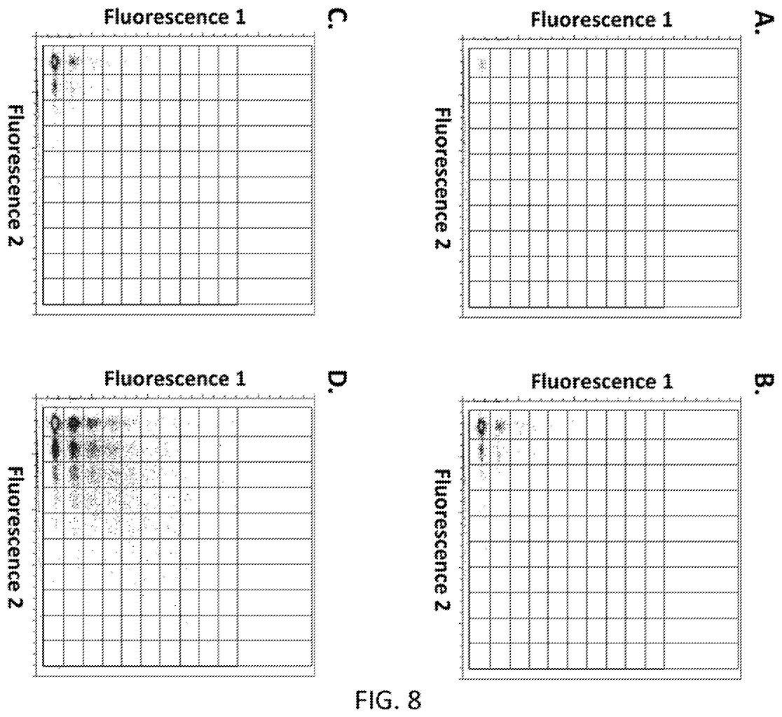

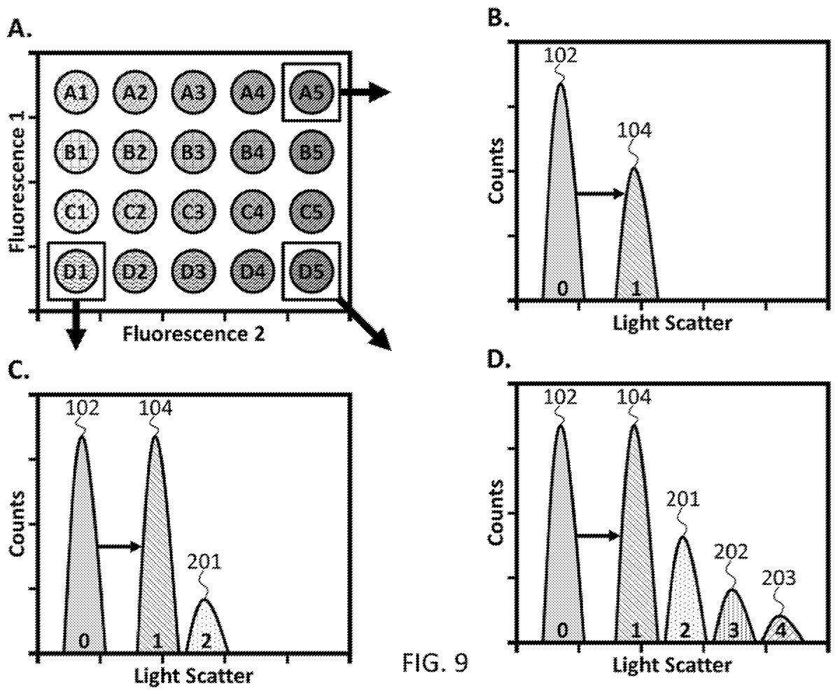

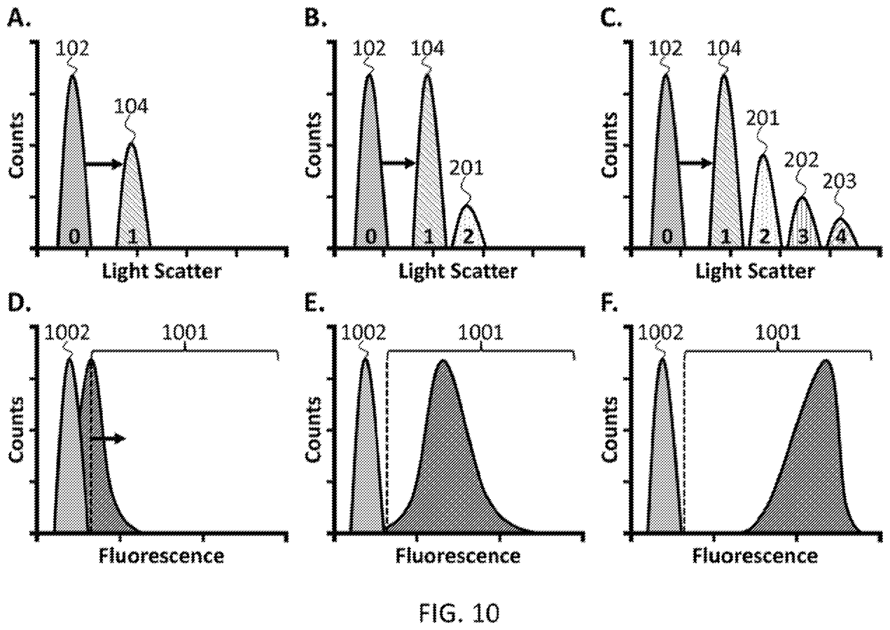

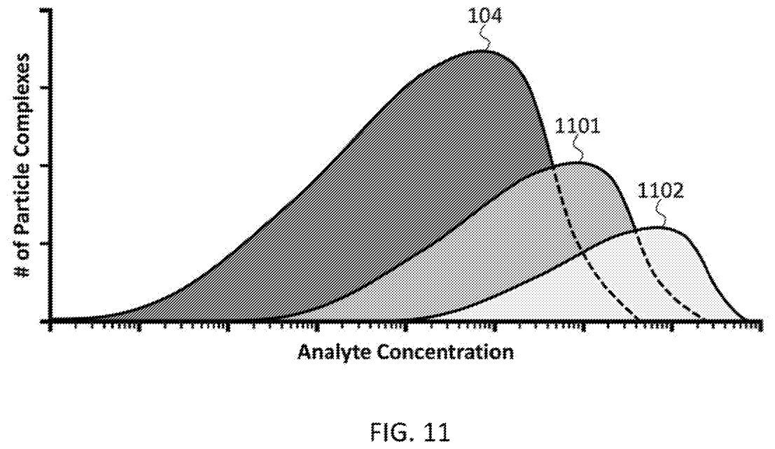

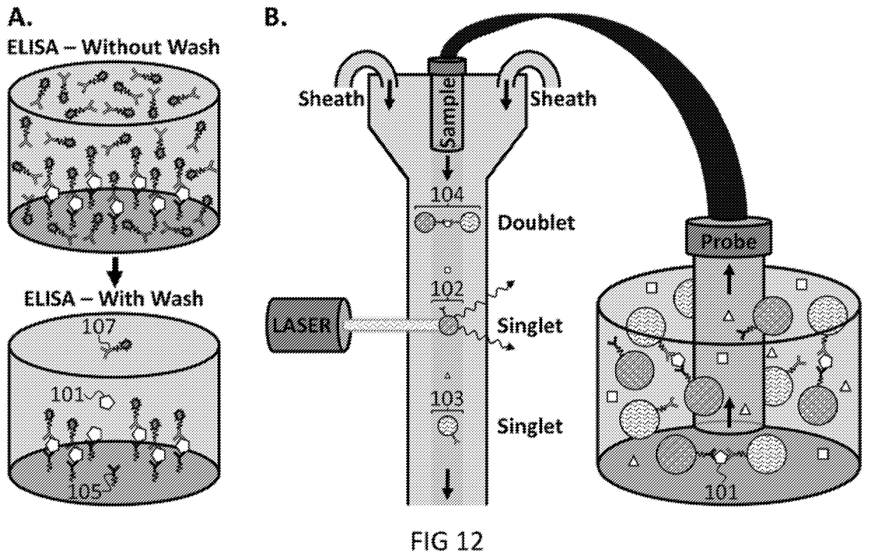

If both the capture and detection particles are fluorescently labeled, and are used at similar concentrations, rather than one being used in excess of the other, then the resulting analyte-linked particle complexes may produce a fractal pattern in the two fluorescent dimensions that are used for labeling. FIG. 7 provides actual preliminary results displaying this phenomena when labeling human prostate-specific antigen (PSA). FIGS. 7A, 7B and 7C respectively present low, intermediate, and high concentrations of PSA. As seen in FIG. 7A, when the concentration of PSA is low, the capture 102 and detection particles 103 form analyte-linked particle doublets 104 when bound to PSA, as normal. However, as the concentration of PSA increases as shown in FIG. 7B and FIG. 7C, higher-order analyte-linked particle complexes are discretely resolved in both of the fluorescence dimensions used. In this case, the analyte-linked particle multiplets in the 2 dimensions, such as 201-1 and 201-2, or 202-1, 202-2 and 202-3, comprise different particle combinations, yet the number of analytes bound per analyte-linked particle complex of the same particle multiple would be equivalent regardless of the specific particle combination. For example, whether an analyte-linked particle triplet is composed of 1×Particle 1+2×Particle 2 (201-1), or vice versa (201-2), both of the analyte-linked particle triplets represent 2 captured analytes. In the magnified inset of FIG. 7C, 202-1, 202-2 and 202-3 each represent analyte-linked particle quadruplets, 203-1 and 203-2 represent two potential combinations of analyte-linked particle quintuplets, and 701 represents one potential combination of an analyte-linked particle sextuplet. These specific particle complexes are provided only as examples and are not intended to be an exhaustive list of the many combinations that can be formed.

Because all of the populations are discretely resolved, each population can be directly enumerated, which can then be used to calculate the analyte concentration either mathematically, statistically, or by comparison to a standard curve. Furthermore, the natural generation of multiple data points at each concentration by this disclosure's technique, unlike many conventional assays that generate only one data point at a given analyte concentration, enables more accurate measurements.

An empirical example of the gating to enumerate the various populations can be seen in FIG. 8, where FIG. 8A, FIG. 8B, FIG. 8C and FIG. 8D respectively represent control, low, intermediate, and high PSA concentrations. In the histograms of FIG. 8, the analyte-linked particle doublets appear at the bottom-left corner of the grid, while the higher-order analyte-linked particle complexes increase in their particle multiple as they progress upward and to the right.

Similar data analyses may be applied to multiplexed assays performed in accordance with this disclosure. FIG. 9 demonstrates one exemplary embodiment of such a multiplexed assay in which fluorescently labeled capture particles are used to simultaneously identify and analyze multiple types of analytes. Specifically, as shown in FIG. 9A, the capture particles are divided into subgroups A1, A2, . . . , D4 and D5. Each subgroup, A1, A2, . . . , D4 or D5, of capture particles is labeled with a unique combination of fluorescent dyes, Fluorescence 1 and Fluorescence 2 that exhibit distinct fluorescence intensities, and is conjugated to a specific reagent targeting a specific type of analyte. For example, the capture particles in subgroup D1 have the lowest intensity in Fluorescence 1 and the lowest intensity in Fluorescence 2; the capture particles in subgroup A5 have the highest intensity in both Fluorescence 1 and Fluorescence 2; and so on. FIG. 9B, FIG. 9C and FIG. 9D illustrate that, once the unbound particles and analyte-linked particle complexes are differentiated and grouped into subgroups according to the fluorescent labels of the capture particles as illustrated in FIG. 9A, then, for each type of analyte, the number of analyte-linked particle complexes containing 0, 1, 2 or more detection particles (102, 104, 201, 202 and 203), and thus analytes, can be easily resolved and enumerated based on the corresponding histograms of light-scatter intensity.

The system described in the preceding exemplary embodiment is not limited to any particular number or variety of fluorophores or spectral labels. In some embodiments of this disclosure, more than two fluorescence channels may be used for analyte labels. In other embodiments, a variety of fluorescence, scatter and/or other optical or spectral properties may be used as analyte labels. Further, the detection particles may also be fluorescently labeled, for example, using colors different from the color of light produced by the capture-particle labels.

The ability to discretely count analyte-linked particle complexes and unbound particles is a major characteristic that differentiates this disclosure from conventional techniques that ultimately rely on analog signal analyses. FIG. 10 highlights schematically the difference in data that may be extracted from experiments using the two approaches. FIG. 10A, FIG. 10B and FIG. 10C depict results using the techniques of this disclosure, while FIG. 10D, FIG. 10E and FIG. 10F are results produced by a conventional analog system. The analyte concentrations increase from left to right in both instances. However, as shown for this disclosure, the number of analyte-linked capture-and-detection-particle doublets (104 in FIG. 10A) increases as the concentration of analyte increases. At intermediate analyte concentrations the number of analyte-linked particle triplets (201 in FIG. 10B) begins to increase. Finally, at higher analyte concentrations higher-order analyte-linked particle multiplets are formed (FIG. 10C). However, in all cases, from FIG. 10A to FIG. 10C, the signals (104, 201, 202, and 203) are always clearly resolved from the background unbound particles 102 and 103. Alternatively, a conventional measurement for extracting analyte concentrations from average intensities poorly resolves the signal (1001) from the background (1002) at lower analyte concentrations (FIG. 10D) and saturates at higher concentrations (FIG. 10F).

Furthermore, as depicted in FIG. 11 as the analyte concentration increases the higher-order analyte-linked particle complexes also exhibit a linear range that is shifted from the initial linear range of the analyte-linked particle doublets. In FIG. 11, analyte-linked particle doublets 104 have an initial linear range that spans several decades, while a particular intermediate-sized analyte-linked particle complex 1101 begins to form at analyte concentrations that are several decades higher and has a linear range that also extends several decades higher than that of the analyte-linked particle doublets 104. Likewise, a particular large-sized analyte-linked particle complex 1102 only begins to form at analyte concentrations that are several decades higher than that required for the intermediate analyte-linked particle complexes 1101, and has a linear range that extends even higher.

Because data gathered using an assay in accordance with disclosure produce many discrete data points, each with their own characteristic population distributions and aggregation behaviors, these data points provide additional characteristics for determining analyte concentrations, and improve the accuracy and reliability of the assay. For example, one common problem exhibited by immunoassays that generate only one data point at a given analyte concentration is that upon reaching a saturation point for the assay they exhibit a hook or prozone effect, where the addition of further analyte inhibits and actually reduces proper antibody binding or complex formation. This leads to uncertainty as to whether any individual result is located on the increasing or decreasing side of the assay signal vs. concentration curve, and requires either performing an additional step after the assay is complete, where more sample is added and it is then reanalyzed, or examining additional dilution points to determine if the signal increases or decreases for the additional data point(s). As demonstrated in FIG. 11, with the primary focus being on the analyte-linked particle doublets in the discrete particle-complex range, higher concentrations simply shift the linear range to proximal higher-order analyte-linked particle complexes, so that multiple data points can be separately quantified and the combination used to accurately determine the analyte concentration.

Unlike conventional assays based on analog signals and molecular reagents, by using the techniques appearing in this disclosure, each analyte-linked capture-and-detection-particle doublet that is enumerated represents a single analyte or analyte complex. Thus, the dynamic range of an assay of this disclosure is directly proportional to the total number of particles counted during an experiment. For example, if 10⁶ particles are analyzed, then the nominal dynamic range would be 6 decades. This would only take minutes to acquire on a basic multi-parameter particle counter, such as a flow cytometer, or an imaging or laser-scanning microscope. In practice, when higher-order particle complexes are taken into consideration, the range could be extended by another decade or more even with the same number of counted particles. Furthermore, since the dynamic range is proportional to the total number of events counted, it could be extended even further by extending the sample acquisition time. For example, acquiring the sample for 10 minutes rather than 1 minute would proportionally increase the dynamic range by another decade. As discussed in the previous embodiments of this disclosure, the practical dynamic range described in this disclosure may be further expanded using a multiplexed assay that combines particle-conjugated and molecular detection reagents in order to respectively measure low- and high-abundance analytes.

FIG. 12 illustrates another significant difference between this disclosure and conventional techniques. In order to accurately measure analyte concentrations, reagent concentrations are commonly used in excess of the target-analyte concentrations in a sample thereby enhancing formation of analyte-reagent complexes in the binding equilibrium. As shown in FIG. 12A, in conventional assays at least one of the reagents is in molecular form (107). Consequently, irrespective of how small the sample volume is, the ubiquitous unbound molecular reagent 107 must be removed from the sample through careful washing before the final measurement. Washing not only consumes time and resources, but it also breaks the equilibrium, resulting in the loss of analyte-reagent complexes thereby biasing results. On the other hand, in this disclosure, both the capture and the detection reagent are conjugated to labeled particles. Without relying on enzymatic or other forms of signal amplification, the analyte-linked particle doublets 104 and higher-order particle multiplets provide a sensitive and reliable probe for their corresponding analyte or analyte complex at the single-analyte level. In addition, since the analyte-linked particle complexes can be readily differentiated from unbound particles 102 and 103 by a multi-parameter particle counter, such as the flow cytometer illustrated schematically in FIG. 12B, no wash is needed to remove the unbound particles before the final measurement. Indeed, simultaneously enumerating the unbound particles enables the concentration of the analyte to be precisely determined using only a fraction of the total sample volume, and provides important statistical and performance-reliability information. Consequently, the binding equilibrium for the assay can be maintained throughout the entire process.

Although the preceding disclosure has been made in terms of various embodiments, it is to be understood that such disclosure is purely illustrative and is not to be interpreted as limiting. Although several exemplary embodiments of this disclosure have been described in some detail, in light of the above teaching, it will be apparent to those skilled in the art that many modifications and variations of the described embodiments are possible without departing from the principles and concepts of the disclosures as set forth in the claims. Consequently, without departing from the spirit and scope of the disclosure, various alterations, modifications, and/or alternative applications of the disclosure will, no doubt, be suggested to those skilled in the art after having read the preceding disclosure. Accordingly, it is intended that the following claims be interpreted as encompassing all alterations, modifications, or alternative applications as fall within the true spirit and scope of the disclosure.

What is claimed is:

1. A system for use in concurrently detecting and quantifying biological analytes, which system upon mixing together forms particle complexes, the system comprising at least one subsystem, wherein the subsystem includes:
a. a set of target analytes, wherein each target analyte is selected from a group consisting of:
i. a single component with multiple binding sites; and
ii. multiple components, each component including at least one binding site;
b. a set of capture particles, each capture particle being capable of binding to a first binding site of the selected target analyte; and
c. a set of detection particles, each detection particle being capable of binding to a second binding site of the selected target analyte that differs from the first binding site of the selected target analyte.

2. The system of claim 1, wherein the capture particles and/or detection particles are labeled with unique physical characteristics.

3. The system of claim 2, wherein particle complexes can form, comprising capture particles and detection particles linked together by one or more of their corresponding target analytes.

4. The system of claim 3, wherein, in each subsystem, the analyte-linked particle complexes and unbound particles can be discretely differentiated and enumerated.

5. The system of claim 4, wherein target analyte refers to any substance whose chemical or biological proper ties are being identified and/or measured; and, wherein particle refers to any small localized object to which several physical, chemical and/or biological properties, such as diameter, charge and/or material composition, can be ascribed.

6. The system of claim 4, wherein the target analyte may bind to a reagent that is conjugated to a capture or detection particle either covalently or using an affinity tag.

7. The system of claim 6, wherein the reagent may be any substance that binds specifically or nonspecifically to a site of the target analyte. Reagents include, but are not limited to: antibodies, binding proteins, peptides, polypeptides, protein complexes, sugars, oligosaccharides, polysaccharides, DNA, RNA, oligonucleotides, polynucleotides, nucleotide complexes, single-stranded nucleic acid sequences, double-stranded nucleic acid sequences, aptamers, natural polymers, synthetic polymers, pharmaceuticals, drugs, lipids, detergents, micelles, liposomes, lipoproteins, extracellular vesicles, exosomes, oncosomes, viruses, virus-like particles, cells, cell fragments, and chemicals.

8. The system of claim 6, wherein the affinity tag refers to any molecule or entity with affinity to, or that can be identified and more generally targeted by, a second molecule or binding partner, and can be used to bring two components, such as a reagent and a particle, together into a complex when differentially conjugated to the pair of components. Affinity tags include, but are not limited to: biotin, streptavidin, avidin, neutravidin, hemagglutinin, poly-histidine, maltose-binding protein, myc, glutathione-s-transferase, FLAG, protein A, protein G, protein L, DNA, RNA, oligonucleotides, polynucleotides, single-stranded nucleic acid sequences, double-stranded nucleic acid sequences, species-specific antibodies, class-specific antibodies, and isotype-specific antibodies.

9. The system of claim 4, wherein a particle may bind directly to an analyte due to conjugating physical, chemical and/or biological properties between the binding partners, including, but not limited to: shape, charge, chemical bonding and/or biological affinities.

10. A system of claim 4, wherein the detection particles in certain subsystems may be replaced with molecular probes.

11. The system of claim 10, wherein the concentrations of the analytes in the subsystems containing molecular probes are measured in accordance with the average number of analyte-linked molecular probes on labeled capture particles.

12. A system of claim 4, wherein, in certain subsystems, modulating agents may be introduced to modulate, inhibit or enhance the binding affinities among the analytes and coupling reagents, or among the components of analyte complexes.

13. The system of claim 4, wherein the subsystems will be differentiated from each other, and the various analyte-linked particle complexes and unbound particles within each subsystem will be discretely detected, differentiated and enumerated, all simultaneously, by any method.

14. The system of claim 4, wherein the subsystems will be differentiated from each other, and the various analyte-linked particle complexes and unbound particles within each subsystem will be discretely detected, differentiated and enumerated, all simultaneously, by their optical, electronic, electromagnetic, fluorescent, radioisotopic, chemical, mass, size, affinity, material composition and/or density signatures, as well as logical combinations of these signatures.

15. The system of claim 4, wherein the subsystems will be differentiated from each other, and the various analyte-linked particle complexes and unbound particles within each subsystem will be discretely detected, differentiated and enumerated, all simultaneously, using a multi-parameter particle counter, such as a flow cytometer, or an imaging or laser-scanning microscope.

16. The system of claim 4, wherein the resulting data from the discrete detection, differentiation and enumeration of a single subsystem will be used to determine the analyte concentration.

17. The system of claim 4, wherein the resulting data from the discrete detection, differentiation and enumeration of multiple different subsystems will be collectively used to determine the analyte concentration.

18. A method for simultaneously detecting and quantifying biological analytes, the method comprising the steps of:
a. selecting a set of target analytes wherein each target analyte is selected from a group consisting of:
i. a single component having multiple binding sites; and
ii. multiple components, each component including at least one binding site;
b. selecting a set of capture particles, each capture particle being capable of binding to a first binding site of the selected target analyte;
c. selecting a set of detection particles, each detection particle being capable of binding to a second binding site of the selected target analyte that differs from the first binding site of the selected target analyte; and
d. mixing the set of the selected target analytes both with the set of capture particles and with the set of detection particles:
i. whereby particle complexes form having capture particles and detection particles bound respectively to analyte binding sites of the capture particles and of the detection particles;
ii. thus linking together at least one capture particle and at least one detection particle by at least one target analyte.

19. The method of claim 18 wherein a unique physical characteristic may label:
a. capture particles; and
b. detection particles.

20. The method of claim 18 wherein the analyte-linked particle complexes and unbound particles can be discretely differentiated and enumerated.

21. The method of claim 20 wherein:
a. the selected target analyte refers to any substance for which at least one property chosen from a group consisting of:
i. chemical property of the selected target analyte; and
ii. biological property of the selected target analyte:
is to be determined from a group consisting of:
i. identifying the property; and
ii. measuring the property; and
b. both the selected capture particles and the selected detection particles respectively exhibit at least one physical, chemical and biological characteristic.

22. The method of claim 20 wherein a reagent is conjugated to one type of particle chosen from a group consisting of:
a. the selected capture particles; and
b. the selected detection particles;
the reagent being bound to the type of particle chosen from the preceding group by a binding mechanism chosen from a group of binding mechanisms consisting of:
a. covalent binding; and
b. an affinity tag; and
the reagent also binding to the binding site of the selected target analyte to which the chosen type of particle binds.

23. The method of claim 22 wherein the reagent is chosen from a group that includes:
a. antibodies;
b. binding proteins;
c. peptides;
d. polypeptides;
e. protein complexes;
f. sugars;
g. oligosaccharides;
h. polysaccharides;
i. DNA;
j. RNA;
k. oligonucleotides;
l. polynucleotides;
m. nucleotide complexes;
n. single-stranded nucleic acid sequences;
o. double-stranded nucleic acid sequences;
p. aptamers;
q. natural polymers;
r. synthetic polymers;
s. pharmaceuticals;
t. drugs;
u. lipids;
v. detergents;
w. micelles;
x. liposomes;
y. lipoproteins;
z. extracellular vesicles;
aa. exosomes;
ab. oncosomes;
ac. viruses;
ad. virus-like particles;
ae. cells;
af. cell fragments; and
ag. chemicals.

24. The method of claim 20 wherein at least one of the selected capture particle and of the selected detection particle binds directly to their respective selected target analytes binding site.

25. A method of claim 20 wherein the detection particles includes molecular probes.

26. The method of claim 25 wherein an average number of molecular probes on labeled capture particles that bind to the target analytes measures a concentration of the target analytes.

27. The method of claim 20 wherein at least one binding affinity within the target analytes particle complexes and coupling reagents included therein is affected by introducing a modulating agent into the mixture in which the particle complexes form.

28. The method of claim 20 wherein simultaneous discrete detection, differentiation and enumeration of various particle complexes and unbound particles by any suitable method differentiates subsystems of various particle complexes from each other.

29. The method of claim 20 wherein simultaneous discrete detection, differentiation and enumeration of various particle complexes and unbound particles is effected by at least one technique chosen from a group consisting of:
a. a technique for measuring optical properties;
b. a technique for measuring electronic properties;
c. a technique for measuring electromagnetic properties;
d. a technique for measuring fluorescent properties;
e. a technique for measuring radioisotopic properties;
f. a technique for measuring chemical properties;
g. a technique for measuring mass properties;
h. a technique for measuring size properties;
i. a technique for measuring affinity properties;
j. a technique for measuring material composition properties; and
k. a technique for measuring density signature properties.

30. The method of claim 20 wherein simultaneous discrete detection, differentiation and enumeration of various particle complexes and unbound particles is effected by a multi-parameter particle counter selected from a group that includes:
a. a flow cytometer;
b. an imaging microscope; and
c. a laser-scanning microscope.

31. The method of claim 20 wherein a concentration of the target analyte is determined using data produced by discrete detection, differentiation and enumeration by a single subsystem of particle complexes.

32. The method of claim 20 wherein a concentration of the target analyte is determined by collectively using data produced by discrete detection, differentiation and enumeration by multiple different subsystems of particle complexes.
